(19) United States
(12) Patent Application Publication
(10) Pub. No.: US 2008/0206285 A1
(43) Pub. Date: Aug. 28, 2008

(54) COMPOSITIONS AND METHODS FOR TREATMENT AND PREVENTION OF LEISHMANIASIS

(75) Inventors: Gerard Papierok, Hyeres (FR); Serge Vicens, Gignac la Nerthe (FR); Jean-Loup Lemesre, Montpellier (FR)

Correspondence Address: MATTHIAS SCHOLL, 14781 MEMORIAL DRIVE, SUITE 1319, HOUSTON, TX, 77079, US

(73) Assignee: BIO VETO TEST (SARL), La Seyne sur Mer (FR)

(21) Appl. No.: 12/053,085

(22) Filed: Mar. 21, 2008

Related U.S. Application Data
(63) Continuation-in-part of application No. 10/480,026, filed on Oct. 11, 2005, now abandoned.
Application No. 10/480,026 is a continuation-in-part of application No. 10/521,922, filed on Aug. 29, 2005.

(30) Foreign Application Priority Data
Jun. 11, 2001 (FR) 01/07606
Jul. 26, 2002 (FR) 02/09506

(51) Int. Cl. A61K 39/008; A61K 39/00; A61P 33/02

(52) U.S. Cl. 424/269.1; 424/184.1

(57) ABSTRACT

Taught herein are therapeutic pharmaceutical compositions (vaccines) and methods for preventing or treating leishmaniasis and infections mediated by intracellular pathogenic micro-organism in mammals and in particular in humans, members of the dog, cat and horse family. The pharmaceutical compositions comprise composition of matter produced by from Leishmania sp. amastigotes and/or promastigotes in a specific germ-free and serum-free medium, and particularly excreted-secreted proteins of Leishmania sp. amastigotes and/or promastigotes.

### CROSS-REFERENCE TO RELATED APPLICATIONS

This application is a continuation-in-part of U.S. patent application Ser. No. 10/480026, filed Oct. 11, 2005, now pending, which is a U.S. National Stage Application of International Patent Application No. PCT/FR2002/001823, filed May 30, 2002, and further claims priority benefits to French Patent Application No. 01/07606 filed Jun. 11, 2001; and is a continuation-in-part of U.S. patent application Ser. No. 10/521922, filed Aug. 29, 2005, now pending, which is a U.S. National Stage Application of International Patent Application No. PCT/FR2003/002358, filed Jul. 25, 2003, and further claims priority benefits to French Patent Application No. 02/09506 filed Jul. 26, 2002. The contents of all of the aforementioned specifications are incorporated herein by reference.

### BACKGROUND OF THE INVENTION

1. Field of the Invention

This invention relates to a specific immunomodulator complex comprising excreted-secreted antigens from Leishmania and its use in prevention and treatment of infections by pathogenic intracellular microorganisms in mammals, and in particular, in humans, canines, felidae, and equidae. More specifically, this invention relates a therapeutic pharmaceutical compositions (vaccines) comprising excreted-secreted products from amastigotes and/or promastigotes of Leishmania sp. produced in a specified axenic and serum-free medium, pharmaceutical compositions described herein being useful for the prevention and treatment of leishmaniasis and infections by pathogenic intracellular microorganisms in mammals, and in particular, in humans, canines, felidae, and equidae.

2. Description of the Related Art

Leishmaniasis comprises a group of parasitic endemic, or even epidemic, infections widespread in the tropical and subtropical regions of the world. The leishmania, flagellate protozoans of the family Trypansomatidae and the genus Leishmania, are the pathogenic agents responsible for the disease. These parasites infect numerous species of mammals, among which humans and dogs comprise the principal reservoirs of the disease. The leishmanias are transmitted to the different hosts during the infecting bite of phlebotomine sandflies. Nineteen species of leishmanias are potentially capable of infecting humans, and depending on the species of leishmanias involved and factors peculiar to the host (genetic, immunological, etc.), they are the source of very diverse clinical manifestations.

Leishmaniasis develops mainly into three distinct clinical forms: cutaneous, mucocutaneous, and visceral depending on whether the parasites affect the mononuclear phagocytic system of the dermis, the mucous membranes, or the internal organs. The cutaneous lesion can remain localized at the point of inoculation of the parasite and correspond to a benign form with spontaneous healing. Besides this form, more serious pathologies exist, caused by disseminated cutaneous leishmaniasis and mucocutaneous leishmaniasis which are very mutilating and disfiguring.

Visceral leishmaniasis affects the mononuclear phagocytic system of numerous organs and tissues, notably the liver, the spleen, and the bone marrow and is fatal in the absence of treatment.

As all vector transmitted diseases, leishmaniasis is characterized by a life cycle that is relatively simple since it is divided between two hosts, mammalian and phlebotomic, and consists of two main forms:
a flagellate form called a promastigote, present in the digestive tract of the phlebotomic vector, where it multiplies prior to acquiring its form that is infectious for the mammalian host, also called the metacyclic form;
a non-flagellate form called amastigote, present in the mammalian host, such as dogs and humans.

Phlebotomine sandflies live in hot regions of the world (hot Mediterranean or tropical climate). To develop, they require a temperature greater than 17° C. (ideal conditions between 22 to 25° C.), a humid atmosphere, and the absence of wind.

Suburban zones of the Mediterranean countries, where the presence of dogs is more sizeable, combine suitable environmental conditions so that the sandflies can reproduce (manure heaps, farms, gardens, wooden shelters, walls, watered lawns, etc.), which promote a larger density of insects near domesticated dogs and humans.

Today, leishmaniasis represents a significant public health problem particularly in developing countries, and they are a subject of study and research both fundamental and applied in particular in the field of immunoprophylaxis. Ninety-seven countries spread over 4 of the 5 continents are affected by leishmaniasis. Threatening some 380 million people throughout the world, these parasites affect approximately 18 million people in the world, with approximately 2 million new cases per year, 90% of these cases being recorded in India, Sudan, and Brazil. Fifteen years ago, the annual global frequency was estimated to be 400,000 cases (300,000 cases of cutaneous leishmaniasis and 100,000 cases of visceral leishmaniasis), with a general incidence of 12 million clinical cases, and a population at risk of approximately 350 million individuals. Currently, the annual global frequency is estimated to be between 1.5 and 2 million new cases per year; of that, 1 to 1.5 million cases represent cutaneous leishmaniasis and 500,000 cases represent visceral leishmaniasis.

Whereas the tropical and subtropical populations are on the front line facing these diseases, the risks of canine and human infection in the Mediterranean basin are often underestimated. Visceral leishmaniasis caused by Leishmania infantum is largely expanded over the different continents of the Old World, and is present in all areas surrounding the Mediterranean basin, the south of France comprising one of the focus areas. Though the vector as well as the parasite present in the south of France appear better adapted to dogs than to humans, the number of human cases of leishmaniasis, currently estimated to be about a hundred cases per year, has been growing fast for the past 10 years, and is being further increased by the ever increasing number of immunodepressed subjects.

Leishmaniasis is also considered to be one of the opportunist diseases of AIDS. Approximately 1500 cases of HIV/Leishmania co-infection have been counted in the south of Europe which represents 90% of the reported cases in the world; Spain is the country the most affected with approximately 60% of these cases. The domestic dog is the main reservoir of the parasite.

Canine leishmaniasis, which is a common pathology of the areas surrounding the Mediterranean, manifests itself in various clinical forms which often lead to the death of the animal. The prevalence of canine leishmaniasis can reach 30% of the canine population in some peripheral urban zones. According to Berrahal et coll. (Am. J. Trop. Med. Hyg. 1996, 55, 273-277), 85% of dogs are PCR (Polymerase Chain Reaction) positive in the endemic zone.

At present, there are no effective immunoprophylactic means against leishmaniasis. The treatment of leishmaniasis calls for pentavalent antimony, pentamidine, pyrazolopyrimidine, amphotericin B, or aminosidine, with a combination of antimony salts-pyrazolopyrimidine being the treatment of choice for canine leishmaniasis. Nevertheless, dogs under treatment remain infectious, in spite of the apparent clinical healing of the animal.

This means that the symptomatic improvement is not correlated to significant reduction of the parasitic load and that there is an epidemiological risk even if clinical healing continues. This situation is further complicated by the emergence of chemoresistance.

At this time, although the problems of chemoresistance considerably complicate treatment, it is still not possible to determine its prevalence in an endemic zone and to diagnose it among patients. Similarly, the molecular bases of this resistance induced in the medically-important stage of the parasite (e.g. amastigote) are still not known.

Finally, the cases of co-infection AIDS/leishmaniasis pose a serious public health problem to the extent that the available therapeutics are less effective among persons sick with AIDS as well as any immunodepressed persons.

No effective vaccine is currently available to combat leishmaniasis and control must be done by chemotherapy. Chemotherapy is unfortunately insufficient due to long, toxic and costly treatments accompanied by numerous cases of relapse and by the emergence of chemoresistance. It is evident, therefore, that the treatment of leishmaniasis over the long term will depend on the discovery of new therapeutic targets and/or vaccines.

Numerous studies concerning the immune responses during experimental murine leishmaniasis have led to the demonstration of the predominant role of cell-mediated immunity and the existence of a duality of the immunological response. There are fundamentally two types of responses against leishmanias: one described “sensitivity”, the other described “resistance”.

The different subpopulations of T lymphocytes (CD4+) limit or exacerbate the infection by means of the lymphokines they secrete. It has thus been demonstrated that the subpopulation of auxiliary T lymphocytes of the Th1 type (producer of interferon gamma and interleukine 2) was capable of eliminating the amastigote intracellular forms by means of the activation of macrophages (Reiner S. L. et al., Annu. Rev. Immunol., 1995, 13, 15 1-1 77. Review). Conversely, the subpopulation of auxiliary T lymphocytes of the type Th2 (producer of interleukine 4) is responsible for exacerbating leishmaniasis.

In humans, certain facts are comparable by nature. In the dog (natural “reservoir” receptive host in the life cycle of L. infantum), the duality of the immunological response is likely. Only one study led by Pinelli et al. (Infect. Immun., 62:229, 1994) on experimental animals naturally infected by L. infantum, made it possible to show that the asymptomatism of the dog (clinical state frequently encountered) is accompanied by the absence of a humoral response and the development of a cell-mediated immunity of the Th1-type with a hypersensitivity reaction of the positive delayed type and elevated rates of interleukine 2 and cachectin (TNF-α) circulating in the biological liquids.

A good vaccine candidate must thus match one or more strongly immunogenic parasitic antigens capable either of blocking the differentiation of the Th2 lymphocytes (Gurunathan S et al., J. Exp Med, 1997 Oct 6, 186, 1137-1 147) (mode of intervention comparable to “desensitization” treatments currently practiced in cases of allergy), or promoting the emergence of the Th1 lymphocytes ensuring the implementation of a protective immunity.

Planning to vaccinate against leishmanias is still problematic today. The attempts have been numerous, but the results are weak and/or contradictory. It can be cited the use of living parasites, irradiated parasites, and completely killed parasites (Moreau Y et al., 1994, Médecine et Armées, 22, 1, 89-93) which have given variable levels of protection among mice and humans.

In the 1980s, purified extracts of parasitic antigens were used in dogs in inducing an exacerbation of the disease: LIF2 fraction and anti-idiotypical vaccine from Dr. Montjour's team. (Chauvy, J “Immunotherapy trails on a canine population in an endemic zone of leishmania” thesis no. 36.1993-Ogunkolade B. W. et al., Vet. Parasitol., 1988, 28, 33-41). Other antigens such as membrane antigens GP63 and lipophosphoglucane (Moreau Y et al., Médecine et Armées, 1994, 22, 1, 89-93) have not produced satisfactory results.

Currently, several molecules are in trials and a final result is pending. One study cites the heat shock protein HSP83 of Leishmania major which stimulates the Th1 method and the protein DP72 (Jaffe. C et al., J. of Immunol., 1990, 144, 699-706). However, none of the current immunization protocols make it possible to obtain a sufficient level of protection, or in any case, the results are not reproducible.

Among the numerous parasites such as Plasmodium, Babesia, Trypanosoma, Toxoplasma, or Shistosoma, it has been shown that excreted-secreted antigens play a predominant role in the establishment of the immune response of the host. The excreted-secreted antigens of leishmanias appear to be involved in the penetration of the macrophage by the parasites, in the inhibition of proteolytic lysosomal enzymes of the macrophage and the negative regulation of the molecules of the Major Histocompatibility Complex (Alexander and Russel, Adv. Parasitol., 1992, 31,175-254).

Moreover, some vaccine approaches conducted in the mouse using excreted-secreted antigens have already been planned with success in different parasitoses (Ouaissi et al., Parasitology, 1990, 100, 115-24; James et al., Trans. R. Soc. Trop. Med. Hyg., 1989, 83, 67-72; Précigout et al., Infect. Immun., 1991, 59, 2799-805; Darcy et al., Ann. Biol. Clin., 1989, 47, 451-7; Capron et al., Mem. Inst. Oswaldo Cruz., 1995, 90, 235-4). But the difficulty in preparation and the numerous serumal and/or cellular contaminants contained in the supernatants of the culture make the use of excreted-secreted antigens difficult in vaccination.

The culture medium described in WO 94/26899 (also described in U.S. Pat. No. 6,458,581) makes it possible to partially solve these problems and to use an abundant, clean and less costly source of the main parasitic stage of leishmanias. The medium is a defined culture medium, which is an axenic, monophasic liquid culture medium devoid of serum. The medium is buffered at the pH of 5.5 to 6.5 and has an osmolarity of at least 400 milliosmoles/kg of liquid for obtaining amastigote forms; and is buffered at the pH of 7 to 7.5 and has an osmolarity of at least 300 milliosmoles/kg of liquid, for obtaining promastigote forms. The culture medium contains further a basic culture medium for insect cells and at least one inorganic salt, a source of amino acids, and a sugar.

### BRIEF SUMMARY OF THE INVENTION

In one embodiment, this invention relates to a pharmaceutical composition comprising an adjuvant and a mixture of excreted-secreted antigens, the mixture being obtained by a process comprising growing promastigotes of Leishmania sp. in an MPm medium.

In a class of this embodiment, the adjuvant is muramyl dipeptide.

In another class of this embodiment, the adjuvant is provided in a weight ratio of between 0.5 and 4 with respect to the mixture of excreted-secreted antigens.

In another class of this embodiment, the composition is provided in a single dose comprising 200 μg of the adjuvant and 100 μg of the mixture of excreted-secreted antigens.

In another class of this embodiment, the process for obtaining the mixture of excreted-secreted antigens comprises further the steps of: (a) filtering a medium in which promastigotes of Leishmania sp. have been grown through a 0.16 μm polyethersulfone microfiltration membrane to obtain a filtrate; and (b) concentrating the filtrate though a 3 kDa polyethersulfone ultrafiltration membrane.

In another class of this embodiment, the mixture of excreted-secreted antigens comprises excreted-secreted antigens having a molecular mass in the range of 52 to 58 kDA, particularly about 55.4 kDa.

In another class of this embodiment, the composition is provided in a formulation suitable for subcutaneous, intradermal, intramuscular, intravenous, parenteral, or oral administration.

In another embodiment, this invention relates to a pharmaceutical composition comprising an adjuvant and a mixture of excreted-secreted antigens, the excreted-secreted antigens being obtained by a process comprising growing amastigotes of Leishmania sp. in an MA1m medium.

In a class of this embodiment, the adjuvant is muramyl dipeptide.

In another class of this embodiment, the adjuvant is provided in a weight ratio of between 0.5 and 4 with respect to the mixture of excreted-secreted antigens.

In another class of this embodiment, the composition is provided in a single dose comprising 200 μg of the adjuvant and 100 μg of the mixture of excreted-secreted antigens.

In another class of this embodiment, the process for obtaining the mixture of excreted-secreted antigens comprises further the steps of: (a) filtering a medium in which amastigotes of Leishmania sp. have been grown through a 0.16 μm polyethersulfone microfiltration membrane to obtain a filtrate; and (b) concentrating the filtrate though a 3 kDa polyethersulfone ultrafiltration membrane.

In another class of this embodiment, the mixture of excreted-secreted antigens comprises excreted-secreted antigens having a molecular mass in the range of 52 to 58 kDA, particularly about 55.4 kDa.

In another class of this embodiment, the composition is provided in a formulation suitable for subcutaneous, intradermal, intramuscular, intravenous, parenteral, or oral administration.

In another embodiment, this invention relates to a method for preventing or treating leishmaniasis comprising administering to a patient the pharmaceutical composition described herein.

In a class of this embodiment, the leishmaniasis is visceral Leishmaniasis.

In a class of this embodiment, the patient is a human or a canine.

In another embodiment, this invention relates to a method for inducing immunostimulation of lymphocytes of a patient in the need of such immunostimulation comprising administering to the patient the pharmaceutical composition described herein.

In another embodiment, this invention relates to a method for inducing immunomodulation of Th1 lymphocytes of a patient in the need of such immunomodulation comprising administering to the patient the pharmaceutical composition described herein.

### BRIEF DESCRIPTION OF THE DRAWINGS

FIG. 1 is a graph showing a number of amastigotes per milliliter of a medium versus culture time for amastigotes grown in the MA1 and MA1m media according to one embodiment of the invention;

FIG. 2 a graph showing a number of promastigotes per milliliter of a medium versus culture time for promastigotes grown in the MP and MPm media according to another embodiment of the invention;

FIG. 3 is a photograph of a gel showing a common epitope of excreted-secreted proteins of promastigotes of various species of Leishmanias (lines 1-2: L. amazonensis, lines 3-4: L. infantum, line 5: L. chagasi);

FIGS. 4a and 4b are photographs of gels illustrating protease activity;

FIG. 5 is a graph illustrating lymphoblastic proliferation versus dosage of administered excreted-secreted proteins of promastigotes;

FIG. 6 is a graph illustrating in vitro parasitemia versus dosage of administered excreted-secreted proteins of promastigotes;

FIG. 7 is a graph illustrating IgG2 response versus dosage of administered excreted-secreted proteins of promastigotes; and

FIG. 8 is a photograph of a gel showing an isolated excreted-secreted antigen of molecular mass of about 55.4 kDa.

### DETAILED DESCRIPTION OF THE INVENTION

This invention relates to immunomodulator compositions that comprise excreted-secreted products of amastigotes and/or promastigotes of leishmanias which suppress Th2-mediated T cell responses and/or promote Th1-mediated T cell response in a reproducible manner

In order to obtain a high yield of the cultivation of amastigotes and promastigotes of leishmanias, media MA1, MA1m, and MP, MPm have been developed, respectively, as follows.

| | | | Medium MPm | |
|---|---|---|---|---|
| | Medium MP for | | (modified) for | |
| | promastigotes | | promastigotes | |
| RPMI-1640 Medium with L | (1.1X) 1000 | mL | (1X) 1000 | mL |
| gluthamine and Hepes | | | | |
| Medium 199 H (10×) modified | 20 | mL | 20 | mL |
| Hemin, bovine | 5 | mg | | |
| Hemin, porcine, irradiated | | | 2 | mg |
| Gentamicine sulfate | | | 0.04 | mg |

| | Medium MA1 | | Medium MA1m | |
|---|---|---|---|---|
| | for amastigotes | | for amastigotes | |
| | (per 800 mL) | | (per 800 mL) | |
| Medium 199 H with Hanks' | 100 | | mL | |
| salts (10×) | | | | |
| CASO Agar | 5 | | g | |
| NaHCO₃ | 0.35 | | g | |
| L-glutamine | 0.75 | | g | |
| HEPES | 5.95 | | g | |
| D-(+)-glucose | 2.50 | | g | |
| H₂O | add to a total of 800 mL | | | |
| Medium 199 H (10×) modified | 4 | | mL | |
| L-glutathione, reduced | 19.7 | | mg | |
| RPMI-1640 Vitamin | 0.16 | | mL | |
| solution (100×) | | | | |
| Hemin, bovine | 4.72 | mg | | — |
| Hemin, porcine, irradiated | — | | | 1.57 mg |
| at 25 kilogray | | | | |
| L-cysteine | 0.29 | g | | — |
| Bathocuproine sulfonic acid | 2.88 | mg | | — |

Medium 199 H with Hanks' salts (10×) contains in g/L: CaCl₂.2H₂O, 1.85; Fe(NO₃)₃.9H₂O, 0.0072; MgSO₄ (anhyd.), 0.9767; KCl, 4.0; KH₂PO₄, 0.6; Na.Acetate (anhyd.), 0.5; NaCl, 80.0; Na₂HPO₄ (anhyd.), 0.4788; DL-Alanine, 0.5; L-Arginine.HCl, 0.7; DL-Aspartic Acid, 0.6; L-Cysteine.HCl.H₂O, 0.0011; L-Cystine. 2HCl, 0.26; DL-Glutamic Acid, 1.336; Glycine, 0.5; L-Histidine.HCl.H₂O, 0.2188; Hydroxy-L-Proline, 0.1; DL-Isoleucine, 0.4; DL-Leucine, 1.2; L-Lysine.HCl, 0.7; DL-Methionine, 0.3; DL-Phenylalanine, 0.5; L-Proline, 0.4; DL-Serine, 0.5; DL-Threonine, 0.6; DL-Tryptophan, 0.2; L-Tyrosine 2Na. 2H₂O, 0.5766; DL-Valine, 0.5; Ascorbic Acid.Na, 0.00056; D-Biotin, 0.001; Calciferol, 0.001; Choline Chloride, 0.005; Folic Acid, 0.0001; Menadione (sodium bisulfite), 0.000016; myo-Inositol, 0.0005; Niacinamide, 0.00025; Nicotinic Acid, 0.00025; p-Amino Benzoic Acid, 0.00005; D-Pantothenic Acid. ½Ca, 0.00001; Pyridoxal.HCl, 0.00025; Pyridoxine.HCl, 0.00025; Retinol Acetate, 0.0014; Riboflavin, 0.0001; DL-α-Tocopherol Phosphate.Na, 0.0001; Thiamine.HCl, 0.0001; Adenine Sulfate, 0.1; Adenosine Triphosphate. 2Na, 0.01; Adenosine Monophosphate.Na, 0.00238; Cholesterol, 0.002; Deoxyribose, 0.005; Glucose, 10.0; Glutathione (reduced), 0.0005; Guanine.HCl, 0.003; Hypoxanthine, 0.003; Phenol Red.Na, 0.213; TWEEN® 80 (Polysorbate 80), 0.2; Ribose, 0.005; and Thymine, 0.003. Medium 199 H with Hanks' salts (10×) was purchased from Gibco BRL, reference 042-01181 of the 1992 catalog.

CASO Agar (also known as Soybean Casein digest Agar; Tryptone Soya Agar; and TSA) contains in g/L: agar, 15; casein peptone (pancreatic), 15; sodium chloride, 5; soya peptone (papainic), 5. CASO Agar has been purchased and is commercially available from Diagnostic Pasteur (France) under the trade name Soybean Casein Digest Agar.

Medium 199 H (10×) modified contains in g/L: CaCl₂.2H₂O, 2.65; Fe(NO₃)₃.9H₂O, 0.0072; MgSO₄ (anhyd.), 0.9767; KCl, 4.0; Na.Acetate (anhyd.), 0.5; NaCl, 68.0; NaH₂PO₄ (anhyd.), 1.22; DL-Alanine, 0.5; L-Arginine.HCl, 0.7; DL-Aspartic Acid, 0.6; L-Cysteine.HCl.H₂O, 0.0011; L-Cystine. 2HCl, 0.26; DL-Glutamic Acid, 1.336; Glycine, 0.5; L-Histidine.HCl.H₂O, 0.2188; Hydroxy-L-Proline, 0.1; DL-Isoleucine, 0.4; DL-Leucine, 1.2; L-Lysine.HCl, 0.7; DL-Methionine, 0.3; DL-Phenylalanine, 0.5; L-Proline, 0.4; DL-Serine, 0.5; DL-Threonine, 0.6; DL-Tryptophan, 0.2; L-Tyrosine 2Na. 2H₂O, 0.5766; DL-Valine, 0.5; Ascorbic Acid.Na, 0.00056; D-Biotin, 0.001; Calciferol, 0.001; Choline Chloride, 0.005; Folic Acid, 0.0001; Menadione (sodium bisulfite), 0.000016; myo-Inositol, 0.0005; Niacinamide, 0.00025; Nicotinic Acid, 0.00025; p-Amino Benzoic Acid, 0.00005; D-Pantothenic Acid. ½Ca, 0.00001; Pyridoxal.HCl, 0.00025; Pyridoxine.HCl, 0.00025; Retinol Acetate, 0.0014; Riboflavin, 0.0001; DL-α-Tocopherol Phosphate.Na, 0.0001; Thiamine.HCl, 0.0001; Adenine Sulfate, 0.1; Adenosine Triphosphate. 2Na, 0.01; Adenosine Monophosphate.Na, 0.00238; Cholesterol, 0.002; Deoxyribose, 0.005; Glucose, 10.0; Glutathione (reduced), 0.0005; Guanine.HCl, 0.003; Hypoxanthine, 0.003; Phenol Red.Na, 0.213; TWEEN® 80 (Polysorbate 80), 0.2; Ribose, 0.005; Thymine, 0.003; Uracil, 0.003, and Xanthine.Na, 0.00344. Medium 199 H (10×) modified was purchased from Flow Laboratories, reference number 14230-54 of the 1992 catalog.

RPMI-1640 Vitamin solution (100×) contains in g/L: D-Biotin, 0.02; Choline Chloride, 0.3; Folic Acid, 0.1; myo-Inositol, 3.5; Niacinamide, 0.1; p-Aminobenzoic acid, 0.1; D-Pantothenic Acid. ½Ca, 0.025; Pyridoxine-HCl, 0.1; Pyridoxine.HCl, 0.1; Riboflavin, 0.02; Thiamine.HCl, 0.1; Vitamin B-12, 0.0005; KCl, 0.2; KH₂PO₄ (anhyd.), 0.2; NaCl, 8.0; Na₂HPO₄ (anhyd.), 1.15. RPMI-1640 Vitamin solution (100×) was purchased and is commercially available from GIBCO BRL under the trade name RPMI-1640 Vitamin solution.

RPMI-1640 Medium with L gluthamine and Hepes (1×) contains in g/L: Ca(NO₃)₂.4H₂O, 0.1; MgSO₄ (anhyd.), 0.04884; KCl 0.4 0.4 0.4 0.4; NaCl, 6.0; Na₂HPO₄ (anhyd.), 0.8; L-Arginine (free base), 0.2; L-Asparagine (anhyd.), 0.05; L-Aspartic Acid, 0.02; L-Cystine. 2HCl, 0.0652; L-Glutamic Acid, 0.02; L-Glutamine, 0.3; Glycine, 0.01; L-Histidine (free base), 0.015; Hydroxy-L-Proline, 0.02; L-Isoleucine, 0.05; L-Leucine, 0.05; L-Lysine.HCl, 0.04; L-Methionine, 0.015; L-Phenylalanine, 0.015; L-Proline, 0.02; L-Serine, 0.03; L-Threonine, 0.02; L-Tryptophan, 0.005; L-Tyrosine. 2Na. 2H₂O, 0.02883; L-Valine, 0.02; D-Biotin, 0.0002; Choline Chloride, 0.003; Folic Acid, 0.001; myo-Inositol, 0.035; Niacinamide, 0.001; p-Aminobenzoic Acid, 0.001; D-Pantothenic Acid. ½Ca, 0.00025; Pyridoxine.HCl, 0.001; Riboflavin, 0.0002; Thiamine-HCl, 0.001; Vitamin B-12, 0.000005; D-Glucose, 2.0; Glutathione (reduced), 0.001; HEPES, 4.77; Phenol Red.Na, 0.0053; NaHCO₃, 2.0. RPMI-1640 Medium with L gluthamine and Hepes (1×) has been purchased and is commercially available from GIBCO BRL under the trade name RPMI-1640 with L gluthamine and Hepes.

The medium MA1m (m for modified) is the medium MA1 without L-cysteine and without bathocuproine sulfonic acid, wherein, bovine hemin has been replaced by porcine hemin, irradiated at 25 kilogray, at a markedly lower concentration (0.003 mM).

For the cultivation of the promastigotes, a reduction in the concentration of 2 components (RPMI and hemin) as well as the addition of an antibiotic (gentamicine) comprise the main modifications of the reference medium MP.

The promastigote or amastigote forms are cultivated in an axenic and serum-free medium.

### EXAMPLE 1

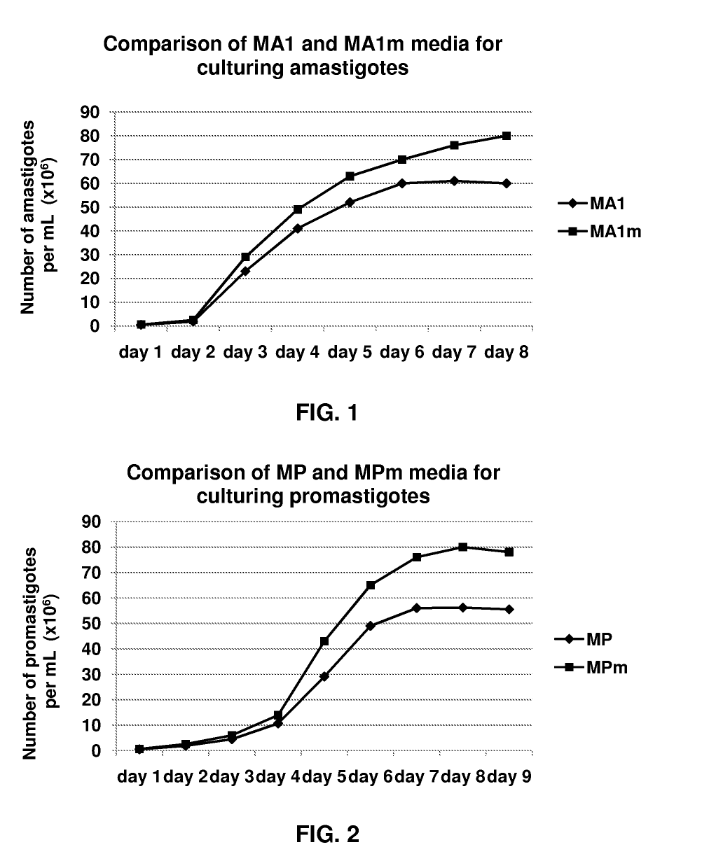

Leishmania infantum MON1 was grown in the media MA1 and separately in MA1m. 1 L of each media was inoculated with 1 mL of culture medium containing 5×10⁵ parasites. The concentration of amastigotes in each of the two media was determined daily over a period of 8 days and compared. (See, FIG. 1). Similarly, Leishmania infantum MON1 was grown in the media MP and separately in MPm. 1 L of each medium was inoculated with 1 mL of culture medium containing 5×10⁵ parasites. The concentration of promastigotes in the two media was determined daily over a period of 9 days and compared. (See, FIG. 2).

After incubation, parasites were eliminated from media MA1m and MPm by tangential filtration through a conventional 0.16 μm polyethersulfone microfiltration membrane, and the individual filtrates were concentrated 100 times by tangential filtration though a conventional 3 kDa polyethersulfone ultrafiltration membrane to obtain parasite-free concentrates comprising excreted-secreted products of Leishmania infantum MON1 grown in each of the media, respectively.

The concentrates were lyophilized and pharmaceutical compositions were prepared by combining an adjuvant with the lyophilized concentrates. The preferred adjuvant was muramyl dipeptide. Other adjuvants could be used as determined by a person skilled in the art.

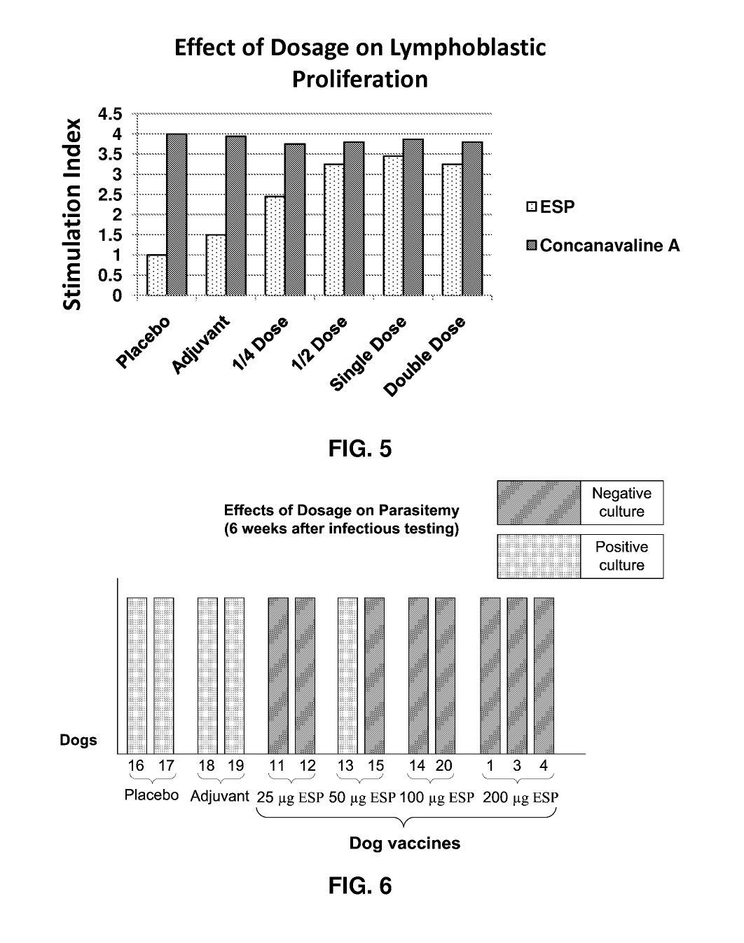

With reference to FIGS. 5-7, an amount of excreted-secreted products in a dose was established according to a dose effect study. A single dose of a pharmaceutical composition particularly comprised about 100 μg of lyophilized excreted-secreted products of Leishmania and 1 mL of a sterile physiological serum.

The composition thus obtained is administered to the infected mammal in the presence of an adjuvant, preferably muramyl dipeptide.

Particularly, the protein/adjuvant ratio is between about 1/0.5 and 1/4 (w/w).

The complex obtained according to the invention comprises products naturally excreted by the promastigotes and/or amastigotes of Leishmanias sp., as well as an adjuvant that preferably induces a cell-mediated response.

Without wishing to be bound by theory, excreted-secreted products of Leishmania comprise excreted-secreted proteins. The excreted-secreted proteins have at least one common epitope carried by one or more major proteins. Their molecular weight varies from 32 kDa to 200 kDa depending on the species of Leishmanias and as a function of the parasitic stage considered.

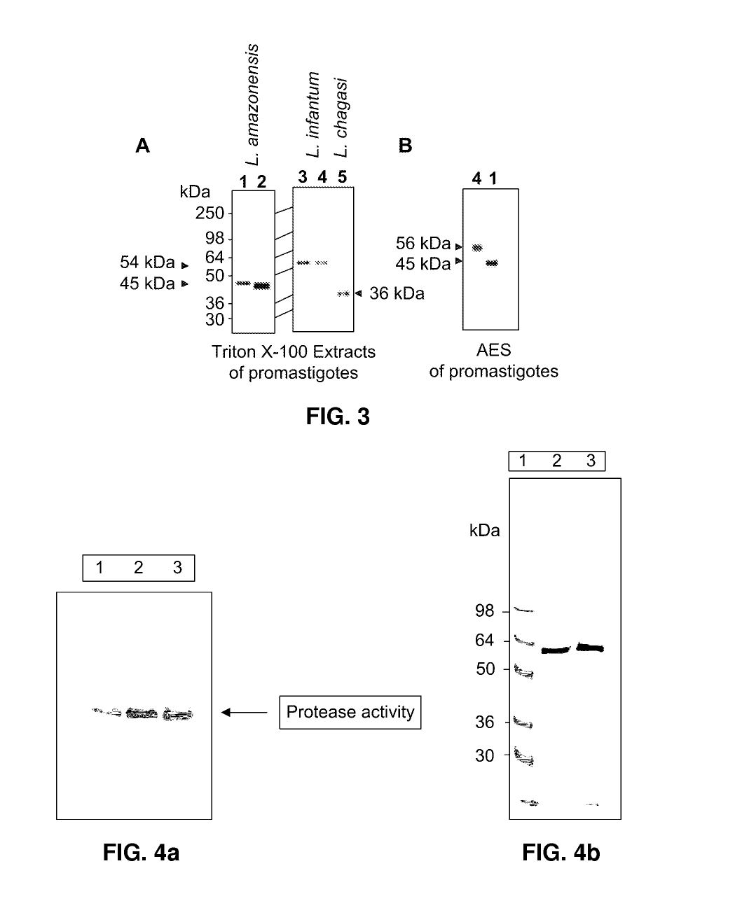

With reference to FIG. 3, a common epitope of various species of Leishmanias was determined by monoclonal antibodies F5. In FIG. 3, A shows TRITON® X-100 (C₁₄H₂₂O(C₂H₄O)₉₋₁₀) extracts of promastigotes, and B shows AES of promastigotes. Lanes marked 1 and 2 correspond to L. amazonensis (45 kDa), lanes marked 3 and 4 correspond to L. infantum (54 kDa), and lane marked 5 corresponds to L. chagasi (36 kDa).

The native proteins that show unidentified protease activity (neither metal, nor serine, nor cysteine protease) are devoid of any serumal or cellular contaminant. With reference to FIG. 4a, lane marked 1 represents control, and lanes marked 2 and 3 represent studied pharmaceutical compositions described herein.

Experiments done in dogs helped determine the optimal dose of the pharmaceutical compositions (vaccines) with a response starting at 100 μg of proteins and 200 μg of muramyl dipeptide.

The specific action mechanism of pharmaceutical compositions described herein prepared according to the invention is verified using the traditional methods that allow the dosage of the proteins, their identification and the measure of their proteasic activity (techniques of Western Blot or immunoblotting and SDS- PAGE) and using more specific methods that show that the innovative therapeutic pharmaceutical compositions described herein acts either by immunostimulation of Th1 lymphocytes, and/or by immunomodulation of the Th2 type in favor of the Th1 type.

Western Blotting makes it possible to individually detect proteins, notably excreted-secreted antigens of amastigote (ESA) and excreted-secreted proteins of promastigote (ESP) by antigen/antibody reaction with the corresponding immunoserums.

For each mammal studied (dog for example), a serologic analysis was done with the ESAs and ESPs.

To prepare the pharmaceutical compositions described herein, proteins are separated at first by discontinuous polyacrylamide gel electrophoresis (PAGE) in the presence of sodium dodecyl sulfate (SDS). This separation is followed by an electrophoretic transfer of proteins to nitrocellulose sheets (Towbin et al. Proc. Natl. Acad. Sci., 1979, 76, 4350-4354). These proteins are then detected by immunoenzymatic reaction by means of an anti-ESP monoclonal antibody. With reference to FIG. 4b which illustrated Western Blot obtained with a monoclonal anti-AES antibody of promastigotes, 4b1 corresponds to marker protein (kDa), 4b2 corresponds to ESP lot to be controlled, and 4b3 corresponds to ESP reference lot.

A parasitologic examination is done on a sample taken directly from a mammal studied, for example, a dog.

A smear of a puncture of the bone marrow was made on a slide. This smear, fixed once by methanol was stained with May-Grünwald-Geimsa and observed by an immersion microscope (1000×).

Samples of the bone marrow are placed into biphasic Novy-MacNeal-Nicolle (NNN) culture medium (Novy and Mac Neal, 1904, J. Infec. Dis., 1:1-30), the liquid phase of which constitutes RPMI 1640 supplemented with 20% decomplemented fetal calf serum. Samples were subcultured every four to six days. The cultures are regularly observed under a two-photon microscope (400×) for 20 min.

The parasitemia was quantified as follows:
+/−: elongated refractive immobile forms;
+: 1 to 5 mobile promastigote forms/field;
++: >5 mobile promastigote forms/field;
+++: culture at confluence.

Evidence of the involvement of a cell-mediated immunity of the Th1 type:

Leishmania promastigotes were cultivated in the culture media defined according to the methods described above. Parasites were harvested at the end of the exponential phase (6-7 days). The parasitic residue was washed three times by centrifugation (2500 g, 15 min, 4° C.) in a PBS buffer. After having verified the viability of the parasites using a vital stain (Trypan Blue), a suspension containing 2×10⁸ parasites per mL was diluted with a PBS buffer containing 0.01% merthiolate (Pinelli et al., 1994, Infect. Immun., 62: 229-235). This constitutes leishmanias for the intradermoreaction test (JDR).

The study of Th1-type immune response that follows was performed on dogs.

Dogs were placed in the lateral decubitus position and shaved in the thoracic zone on an area behind the elbow approximately 5 cm by 10 cm in size. Four circles 10 mm in diameter were marked using a felt-tip pen.

Into the center of the circles, 0.1 mL of solution is injected by an intradermal injection. Two circles receive the solution of leishmanias and the two other circles received a saline solution as a negative control. Reading of the Intra Demo Reaction (IDR) was done 48 hours later.

The test was considered positive if the mean of the two observed induration diameters was greater than or equal to 5 mm. The observation of an erythema without induration was considered a negative (Pinelli et al., 1994, Infect. Immun., 62: 229-235; Marty et al., 1994, Trans. Roy. Soc. Trop. Med. Hyg., 88, 658-659).

Next, a test was done to assess lymphocyte proliferation.

Peripheral blood mononuclear cells (PBMC) of dogs were isolated by Ficoll gradient (density 1.078) by centrifugation at 800 g for 20 min at ambient temperature. These cells were cultivated on a plate having 96 wells at a concentration of 2×10⁵ cells per well in the presence of 2 μg per mL of Concanavalin A (Sigma), and 5 μg per mL of ESP or 20 mL of supernatants of the culture harvested in the stationary phase of growth of promastigotes (SP) per well, and in the absence of any additive in a volume of 200 mL of the medium RPMI 1640 supplemented with 5% decomplemented fetal calf serum, 2 mM of L-glutamine, 100 U of penicillin per ml, and 100 mg of streptomycin per mL. The optimal antigen and mitogen concentrations have been determined in prior experiments. The PBMCs are incubated for 72 hours in a humid atmosphere at 37° C. in the presence of 5% CO, then for 20 hours with 0.5 μCi of ³H thymidine. The cells were harvested over a filter and the incorporation of the radioactivity was determined by counting in a scintillating liquid (P-counter). All of the tests were performed in triplicate.

A more rapid and more sensitive immunohistochemical method using BrdU (5-bromo-2′-desoxyuridine), a structural analog of thymidine, was also used to measure cellular proliferation (BrdU, cell proliferation detection kit III, Boehringer Mannheim, Germany). The BrdU was added for 18 hours after 72 hours of incubation. The cells which have incorporated the BrdU in their ADN are easily detectable in the presence of a monoclonal BrdU antibody.

Proliferative responses were expressed as stimulation indices that represent the ratio of the average proliferation after stimulation to the mean proliferation in the absence of antigen.

The lymphocyte proliferation has also been estimated by visual readings under a photon microscope (−: negative; +/−: slight proliferation; +: little proliferation less than 5 points per microscopic field; ++: mean proliferation greater than 5 points; +++: strong proliferation).

The titration of the leishmanicidal activity of the monocytes was done according to the Lemesre method described below.

For this test, the monocytes and lymphocytes were isolated from the venous blood of dogs. The monocytes were cultivated for 3 days at the rate of 10⁵ cells per well in the culture chambers (Labteck) in the RPMI 1640 complete medium (containing 25 mM HEPES, 2 mM L-glutamine, 100 U penicillin per ml, 100 mg streptomycin per mL and 10% inactivated fetal calf serum) at 37° C. in a humid atmosphere containing 5% CO₂. After 3 days of cultivation, the macrophages were washed with RPMI complete medium, supplemented with fresh medium and put in contact with the metacyclic promastigote forms of L. infantum in a ratio of 5 parasites per cell, at 37° C. for one night or for 5 hours. The macrophages were then washed with fresh RPMI complete medium in order to eliminate non-phagocytic parasites. The cells were put in incubation either alone, in the presence of 5 μg of ESP antigens, or in the presence of autologous lymphocytes, or in the presence of supernatants of the co-culture of infected macrophages and autologous lymphocytes and corresponding controls (harvested at 5 hours) and this was done at 37° C. in a humid atmosphere of 5% CO₂, for the duration of 48 hours. When they were used, the lymphocytes cultivated separately were washed, counted, and added to the macrophages in the ratio of 2 lymphocytes per macrophage.

After 48 hours of incubation, the cells were washed three times in a PBS buffer 0.01 M, pH 7.2, fixed with methanol then stained with Giemsa. The leishmanicidal activity of the macrophages was estimated under a two-photon microscope (1000×). For determining the percentage of macrophages infected and the number of intact amastigote forms per 100 cells, 2 times 200 cells were observed in duplicate. The results were expressed as percentage inhibition of the parasitic index=100−(IP×100).

IP=parasitic index=((mean number of amastigotes per macrophage in the treated sample)×(mean percentage infected macrophages in the treated sample))/((mean number of amastigotes per macrophage in the control sample)×(mean percentage infected macrophages in the control sample)).

One can also monitor the concentration of nitrogen monoxide (NO) to ascertain the destructive activity of the monocytes against the Leishmanias. The synthesis of NO by the monocytes is in fact sign of the destruction of the leishmanines by monocytes that have been activated by the cytokines of the interferon gamma type (IFNγ).

NO has a high chemical reactivity. In the presence of water and oxygen, this molecule is rapidly oxidized in a stoichiometric manner and forms the nitrites (NO₂⁻) according to the following reaction:

4NO^{o}+O₂+2H₂O→4NO₂⁻+4H⁺

The nitrites accumulate in the media and are easily detectable chemically by the Griess method.

To 50 μL of supernatant to be tested, 60 μL of Griess A (sulfanilamide 1% in HCl 1.2N) is added and 60 μL of Griess B (N-(1-napthtyl)ethylenediamine 0.3%) were added. The colorimetric reaction develops in the dark for 2 minutes. The optical densities obtained at 540 nm are corrected by the subtraction of the ODs obtained on the wells containing only the culture medium.

The values obtained are recorded on a calibration curve (OD=f(NO₂)) made from the known concentrations of NO₂⁻.

The table below shows the serologic responses obtained during the experiments and the monitoring of the parasitemia (analysis made 2 months and 8 months after the infectious test).

| | | | | | | PARASITEMIA | |
|---|---|---|---|---|---|---|---|
| | | Quanti- | SEROLOGY | | | (on marrow | |
| | | tative | | | ELISA | puncture) | |
| | | immun- | Western | Western | (IgG2) | | Cultivation |
| | | ofluo- | Blotting | Blotting | ESA/ | Direct | on NNN |
| | Dogs | rescence | (ESA) | (ESP) | ESP | Exam | medium |
| Control | MUMA | — | − | − | − | + | ++ |
| Dogs | LEO | — | − | − | − | + | ++ |
| Dogs | LOUBARD | 1/200 | + | + | + | − | − |
| immunized | | | | | (0.700) | | |
| with | MINA | 1/200 | ± | ± | + | − | − |
| ESA | | | | | (0.450) | | |
| Dogs | NOUGAT | 1/800 | + | + | + | − | − |
| immunized | | | | | (0.780) | | |
| with | MINON | 1/100 | ± | ± | + | − | − |
| ESP | | | | | (0.520) | | |

Key: Immunofluorescence (considered positive if the titer is [symbol] 1/100); ELISA: Cut off = 0.300 OD (optical density); Parasitemia: cultivation on medium NNN: − = absence, ++ = more than 5 mobile promastigote/field

The following table shows the cellular type responses obtained and the inhibitor role of the serums on the parasitic proliferation (analysis made 2 months after the infectious test).

| | | | | | | | % INHIBITION | |
|---|---|---|---|---|---|---|---|---|
| | | CELL-MEDIATED RESPONSES | | | | | OF LEISHMANIAS | |
| | | | | | Leishmanicidal | | PROLIFERATION | |
| | | | Test for | | activity | Dosage | Proliferation | Proliferation |
| | | | lymphoblastic | | of the | of NO | of the | of the |
| | Dogs | IDR | proliferation | | monocytes | (in μM) | promastigotes | amastigotes |
| Dogs | MUMA | + | + 2.1 | (3) | 15.5% | 0.3 | 20% | 15% |
| Controls | LEO | − | + 1.2 | (3.1) | 21.5% | ND | ND | ND |
| Dogs | LOUBARD | + | ++ 2.9 | (3.2) | 58.9% | ND | 50% | 41% |
| immunized | MINA | + | ++ 3.8 | (4.2) | 47.8% | ND | 69% | 52% |
| with ESA | | | | | | | | |
| Dogs | NOUGAT | + | ++ 3.1 | (4.2) | 75.6% | 3.9 | 98% | 54% |
| immunized | MINON | + | +++ 3.5 | (4.5) | 64.1% | 2.8 | 72% | 56% |
| with ESP | | | | | | | | |

Key: IDR: The intra-dermo reaction test is considered positive (+) if the induration is ≧ 5 mm 48 h after intradermal injection; Lymphoblastic proliferation test: The results are expressed by a reading in photon microscope and in stimulation indices (between parentheses, stimulation index of the control + Concanavalin A): +: weak proliferation, ++: medium proliferation, +++: strong proliferation; Leishmanicidal activity of the monocytes: expressed as a percentage of inhibition of the parasitic index; Dosage of NO: Inhibitor role of the serums: results expressed as a percentage inhibition of growth; ND = Not determined

Inhibitor role of the excreted-secreted antibodies anti-factors on the parasitic development of L. infantum:

These tests intend to show the possible inhibitor effect of the anti-ES antibodies on the proliferation and differentiation in vitro of the parasites.

100 μL of immune serum previously inactivated (56° C. for 45 minutes) from different groups of dogs were placed in contact with 5×10⁶ metacyclic promastigote forms for thirty minutes at ambient temperature. Viability tests before and after treatment (see above) were done to establish mortality. The parasites treated this way were cultivated either at 25° C. in the RPMI 1640 medium containing 10% FCS (fetal calf serum), or at 37° C. in the MAA120 medium (10⁶ parasites per mL of medium). The kinetics of proliferation of the promastigote forms and the kinetics of the amastigote forms were established by daily counting of the cells under a microscope. The results were expressed as percentage inhibition of growth.

The innovative character of the pharmaceutical compositions described herein lies not only in the induction of a specific cellular response of the Th1-type, but also in the production of low antibody rates that are very effective towards promastigotes and amastigotes of Leishmania.

For the procedure of the studies, other specific techniques were used.

Method of infectious examination

The infectious examination consists of injecting intravenously to a healthy dog a mixture of 10⁶ treated metacyclic promastigotes and 5×10⁶ peritoneal macrophages of a healthy dog, infected in vitro by amastigotes. The promastigotes and infected macrophages are combined and diluted with sterile physiological serum to a final volume of 1.5 mL just prior to injection.

Detection of immunoglobulins of the type G2 (IgG2) of dogs, specifically of the ES

This detection is done by the Western Blot method while using a conjugate anti-IgG2 (immunoglobulins G2) of dog, and by the ELISA method according to the microtitration technique of Kweider et al. (J. Immunol. 1987, 138, 299).

The pharmaceutical compositions according to the invention can be administered in various ways, and particularly by subcutaneous injection, by intradermal injection, by intramuscular injection, or orally. Other administration methods can be used, including parenteral or intravenous administration.

In a general manner, a pharmaceutical composition (vaccine) is formulated in an injectable form comprising a lyophilized fraction that is combined with a liquid fraction (diluents).

The doses used for prevention and immunotherapy are different, and vary depending on the mode of injection. If the mode of administration is subcutaneous or intramuscular injection, a full dose (100 μg of excreted-secreted proteins and 200 μg of adjuvant) is administered to a dog regardless of race, age, and sex for a preventative effect, and a half dose (50 μg of excreted-secreted proteins and 100 μg of adjuvant) is administered to a dog regardless of race, age, and sex for immunotherapy of leishmanian dogs. If the mode of administration is intradermal injection, a half dose is administered in dogs for a preventative effect, and a quarter dose s administered in leishmanian dogs for a therapeutic effect.

The methods of injections are described further in the examples of immunotherapy and vaccination as well as in the toxicity studies.

Toxicity studies on pharmaceutical compositions described herein were performed on 30 dogs.

All the dogs were adult beagles between 1 year to 6 years old, 50% male and 50% female, sourced from a non-endemic zone. These dogs were perfectly healthy, and had a serology and a Intra Demo Reaction (IDR) test negative for Leishmania.

Among these dogs, some received placebos. Parallel to the clinical monitoring, a monitoring of the specific immune system status with regard to pharmaceutical compositions described herein was performed (demonstration of the induction of the humoral and cell-mediated immunity of the Th1-type, only in vaccinated dogs).

Protocols

The tests were done with Good Laboratory Practices (GLP) and Good Clinical Practices (GCP).

| | | | | | |
|---|---|---|---|---|---|
| Primo | → 4 weeks of | → Dose | → 4 weeks of | → Overdose | → 4 weeks of |
| vaccination | observation | Repeated | observation | (2 simultaneous | observation |
| | | | | doses) | |

The pharmaceutical compositions (vaccines) were injected subcutaneously.

Tolerance monitoring:

After administration, a direct visual and palpatory examination for pain, tumefaction, heat, and pruritus was performed every day for 14 days from the time of injection.

A monitoring of the general tolerance was also performed. This involved a rapid daily examination with taking of the temperature, a weekly clinical veterinary examination including a ganglionic palpation (popliteal), an abdominal palpation, monitoring for arthritis and uveitis and weighing.

The hematological and biochemical monitoring (creatinine, urea, transaminases) was performed 3 weeks after eacn injection.

Toxicity

Among the 30 dogs, including 7 placebos, no general disorders were observed. Only a few local minor reactions were reported: slight edema at the point of injection, moderate erythema and slight pruritis. These problems are benign and spontaneously disappear in 24 to 48 hours. They are quite consistent with a cell-mediation vaccine.

No anomaly was noted at the hematological and biochemical level. Similar results were obtained after intradermal injection in 5 dogs and after intramuscular injection in 5 dogs.

Thus, the pharmaceutical compositions described herein is deemed to be safe.

Specific Activation of the T lymphocytes of the Th1-type

In parallel with serological monitoring by traditional immunofluorescence using strips coated with promastigotes (serological reference method for canine leishmaniasis) which turned out to be low for all studies dogs, a study of the cell-mediated response via lymphoblastic proliferation and via leishmanicidal activity of monocytes was performed on a sample of 30 dogs, incl. 7 placebos.

The 7 placebos did not induce a cellular response specific to the vaccine antigen. On the contrary, the 23 dogs vaccinated indeed had an induction of the Th1 system with notable lymphocyte proliferation indexes specific to pharmaceutical compositions described herein comparable with the control index (ConcanavalineA), which accompanies elevated parasitic inhibition percentages (>40% with a mean of 60% on 23 vaccinated dogs).

Thus, the pharmaceutical compositions described herein have the effect of inducing lymphocytes to activate monocytes towards leishmania while having no effect on the Th2 system.

Dose effect study of the pharmaceutical compositions

This experiment had the goal of determining the minimum dose of pharmaceutical compositions (vaccines) which induces an effective Th1 response.

For this purpose, 12 adult beagles between 1 and 6 years old from a non-endemic zone were divided into 6 groups of 2, as follows: 1st group: placebo; 2nd group: placebo+200 μg of adjuvant; 3rd group: 25 μg excreted-secreted proteins and 50 μg of adjuvant; 4th group: 50 μg excreted-secreted proteins and 100 μg of adjuvant; 5th group: 100 μg excreted-secreted proteins and 200 μg of adjuvant; and 6th group: 200 μg excreted-secreted proteins and 400 μg of adjuvant.

These tests were performed with Good Laboratory Practices (GLP) and Good Clinical Practices (GCP).

| | | | | |
|---|---|---|---|---|
| Primo | → 4 weeks of | → 2^{nd} | → 4 weeks of | → Infectious |
| vaccination | observation | injection | observation | vaccination |
| | | | | test |

The infectious test consisted of infecting the dogs intravenously injecting metacyclic promastigotes and monocytes infected with amastigotes.

Following the 2^{nd} injection, the study of the immune state confirmed that the dogs that received the pharmaceutical compositions (vaccine) were indeed in Th1-state. The onset of maximum response was observed starting with a dose of 50 μg of excreted-secreted proteins being injected. This level phenomenon was observed both in the lymphocyte proliferation test and for the monocyte activity.

FIG. 5 shows results of the dose effect study: lymphoblastic proliferation study according to the injected vaccine dose.

On the other hand, a parasitemic study on the marrow puncture was done 2 months after the infectious test using the culture reference medium NNN (Novy and MacNeal, J. Infect, Dis, 1904, 1, 1-30).

4 placebo dogs and a dog having received 50 μg of excreted-secreted proteins exhibited positive parasitemia.

FIG. 6 shows results of the dose-effect study: study of the parasitemia, 6 weeks after the infectious test according to the injected vaccine dose.

Specific antibodies linked to the Th1 system

As previously shown, the Th1 state corresponds to a cell-mediated response with an activation of the macrophages via the lymphocyte producers of specific cytokines. This is the main role of the pharmaceutical compositions described herein. This cellular response is accompanied by a low humoral response that can be easily demonstrated by the traditional method of immunofluorescence using a conjugate anti IgG total marked by fluorescein.

Nevertheless, certain preliminary work among humans (Kawano P et al, Parasite Immunol., 1995, 17, 451-458) and in dogs (Nieto C. G et al, Vet Immunol and Immunopathology, 1999, 67, 117-130) shows that the IgG isotypes would be markers of the immune dichotomy Th1/Th2. More specifically, a dog suffering from leishmaniasis with conclusive clinical signs has a high level of antibodies mainly of the isotype IgG1, while an asymptomatic dog has antibodies specific to the isotype IgG2. Dogs that received pharmaceutical compositions described herein have low levels of IgG2 specific to excreted-secreted proteins, which is in keeping with the preferential expansion of T lymphocytes of the Th1 type.

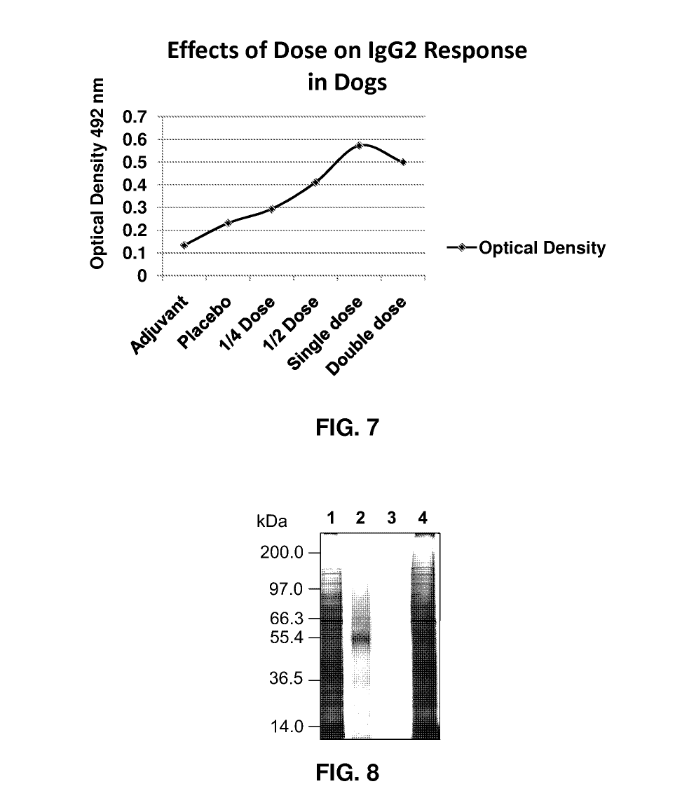

FIG. 7 shows the specific response in vaccinated dogs of IgG2 towards pharmaceutical compositions versus dose (ELISA method on test wells).

Immunotherapy results

According to Pinelli (Pinelli E. et al., Infect Immun., 1994, 62, 229-235), resistance and susceptibility to Leishmania is associated with the development of strong Th1 and Th2 responses, respectively.

An increased production of antibodies corresponds to hyperproteinemia and induces the appearance of immune complexes that cause a renal problem (increase in the creatinine and blood urea).

During the studies and trials, an attempt was made to modulate towards a Th1 state by administering to the leishmanian dogs intramuscular doses of pharmaceutical compositions described herein. The monitoring of the immune state and the clinical observation were done before and after treatment.

### EXAMPLE 2

A male dog of the British spaniel breed, named LOYD, age 6, living lived near Aix en province in the middle of the endemic zone, spent the majority of his time outdoors, and was predisposed to be bitten by sand flies. LOYD exhibited numerous cutaneous lesions accompanied with a general state of fatigue and thin appearance, all reminiscent of a leishmaniasic canine. The cutaneous lesions were of many types: pustules and papules at the level of the nose; erythema on the side and on the face inside the ears; pruritis, squama and scabs at the level of the elbows.

A veterinarian diagnosed LOYD with pemphigus foliaceous accompanied by leishmaniasis. This latter was confirmed by a direct observation in a microscope of leishmanias from a cutaneous tracing and a serological analysis which gave a Leishmania-positive serum titer of 1:1600 using immunofluorescence.

For 8 months, a traditional treatment with antimony salts and corticoids was administered but didn't eradicate leishmaniasis. Then, immunotherapy was administered which consisted of 4 intramuscular injections of the pharmaceutical composition described above comprising 50 μg (½ dose) of excreted-secreted proteins of promastigotes, the injections being 10 days apart.

The analysis of the immune state prior to injections confirmed that the dog was in Th2-dominated immune state with no lymphoblastic proliferation tests and monocyte activation.

A week after the second injection, LOYD regained his appetite and a certain amount of vitality. A slight cutaneous improvement was observed. One month after the last injection, LOYD regained normal clinical appearance with a notable increase in weight and disappearance of 80% of cutaneous lesions.

Analysis of the immune state by immunofluorescence confirmed a reduction in the anti-leishmania antibody titer which dropped to 1/400. In parallel, monocyte activation was established with 75% inhibition of the parasitic index and the lymphoblastic proliferation test was fully positive.

A study of the parasites by cultivation on the NNN medium was negative. 8 months after the beginning of treatment, LOYD only exhibited lesions on the nose which corresponded to pemphigus foliaceous. The lesions disappeared after corticoid treatment. The biological analysis confirmed that LOYD was inTh1-dominated immune state.

### EXAMPLE 3

A male dog of the Rottweiler breed, named JAZZ, age 5, had clinical signs specific to leishmaniasis, including the presence of numerous shiny squama, right periocular hair loss, ulcerous lesions at the level of the 2 front elbows, and a pronounced state of fatigue. Serological analysis gave a Leishmania-positive serum titer of 1:400 using immunofluorescence confirming the clinical diagnostic.

Immunotherapy was administered and consisted of 3 intramuscular injections of the pharmaceutical composition described above comprising 50 μg (½ dose) of excreted-secreted proteins of promastigotes, the 3 injections being 10 days apart. The analysis of the immune state prior to injections showed that the dog was in Th2-dominated immune state_with a greatly positive parasitemia (bone marrow).

One month after the last injection, clinical signs of leishmaniasis had retroceded with a notable healing of the ulcerous lesions, sizeable disappearance of the squama, and nearly fully-regrown periocular hair. Serological analysis still gave a Leishmania-positive serum titer of 1:400 using immunofluorescence.

However, analysis of the cellular response confirmed that JAZZ was in Th1-dominated immune state with a positive lyrnphoblastic proliferation test and high intramacrophagic leishmanicidal activity. The parasitemia was negative (cultivation of the bone marrow in a NNN medium).

Accordingly, it is safe to say that the therapeutic pharmaceutical compositions described herein promote passage from Th2-dominated immune state to Th-1 dominated immune state that leads to healing, and promote sizeable production of antibodies.

Pharmaceutical composition administration results (vaccination results)

Protocols

In order to evaluate the efficacy of pharmaceutical compositions described herein for preventing leishmaniasis, tests were carried out on 6 perfectly healthy dogs. The dogs had a negative leishmaniasic serology, a negative parasitemia as well as fully negative cellular response tests specific to Leishmania.

The dogs inhabited environment free from sand flies. The dogs were divided into 3 groups, each group comprising a male and a female dog, as follows. Control group (placebos): Negative control: LEO, Pointer breed, male, 3 years old. Sole adjuvant control: MUMA, British spaniel breed, female, 6 years old. Group of dogs vaccinated with excreted-secreted proteins of promastigotes (ESP): MINON, Weimaraner breed, female, 2½ years old. NOUGAT, Pointer breed, male, 2½ years old. Group of dogs vaccinated with excreted-secreted antigens of amastigotes (ESA): LOUBARD, British spaniel breed, male, 4 years old. MINA, Weimaraner breed, female, 3 years old.

The administration of pharmaceutical compositions according to the invention (vaccination) scheme was as follows:

| Day 0 | | | | Day 28 | | | | Day 84 |
|---|---|---|---|---|---|---|---|---|
| 1^{st} injection | → | 4 weeks | → | 2^{nd} injection | → | 8 weeks | → | infectious |
| 1 subcutaneous | | | | 1 subcutaneous | | | | test |
| dose | | | | dose | | | | |

The dogs were clinically monitored every two weeks. The biological analysis was performed as follows:

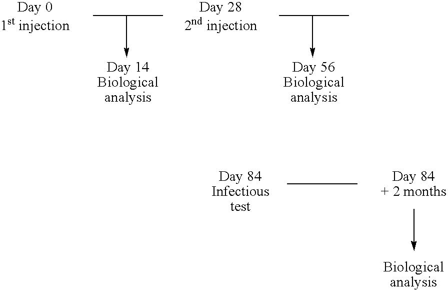

The biological analysis consisted of:
biochemical analyses: urea, creatine, transaminases
hematological analyses: count, formula
serology leishmaniasis: quantitative anti-Leishmania immunofluorescence by the Western Blot method towards excreted-secreted antigens and dosage by the ELISA method of specific IgG2s.
cellular response tests: lymphoblast proliferation test, study of the activation of macrophages and IDR test (IntraDermoReaction), dosage of NO.
study of the neutralizing role of the anti ES antibodies.

Additionally, search for leishmania parasites was performed by cultivation on medium NNN from bone marrow after the infectious test and direct observation under a microscope.

Results

Clinical monitoring

No significant clinical manifestation appeared during this study. A slight weight loss and the appearance of squama in one dog (LEO) was observed 2 months after the infectious test.

Biological monitoring

The biochemical and hematological parameters were normal during this study.

Leishmaniasis serology and parasitemia

Prior to injections, the dogs exhibited negative serologies and parasitemias. The following table shows the serological responses obtained during the experiments performed and the monitoring of the parasitemia. Analyses were made 2 months and 8 months after the infectious test.

| | | | | | | PARASITEMIA | |
|---|---|---|---|---|---|---|---|
| | | Quanti- | SEROLOGY | | | (on marrow | |
| | | tative | | | ELISA | puncture) | |
| | | immun- | Western | Western | (IgG2) | | Cultivation |
| | | ofluor- | Blotting | Blotting | ESA/ | Direct | on NNN |
| | Dogs | escence | (ESA) | (ESP) | ESP | Exam | medium |
| Control | MUMA | — | − | − | − | + | ++ |
| Dogs | LEO | — | − | − | − | + | ++ |
| Dogs | LOUBARD | 1/200 | + | + | + | − | − |
| immunized | | | | | (0.700) | | |
| with | MINA | 1/200 | ± | ± | + | − | − |
| ESA | | | | | (0.450) | | |
| Dogs | NOUGAT | 1/800 | + | + | + | − | − |
| immunized | | | | | (0.780) | | |
| with | MINON | 1/100 | ± | ± | + | − | − |
| ESP | | | | | (0.520) | | |

Key: Immunofluorescence (considered positive if the titer is [symbol] 1/100); ELISA: Cut off = 0.300 OD (optical density); Parasitemia: cultivation on medium NNN: − = absence, ++ = more than 5 mobile promastigote/field

Only the immunized dogs had antibodies specific towards ESA and ESP (Western Blot), the specific IgG2s (ELISA) and negative parasitemias. A slight appearance of total antibodies (1/200 by IF) was noted in all of the dogs after the infectious test.

Only the control dogs (LEO and MUMA) had positive parasitemies and absence of specific antibodies IgG2 anti ES.

Cell-mediated response

Before injections, the dogs had a fully negative cell-mediated response to Leishmania infantum. As shown below, only the immunized dogs had positive lymphoblastic proliferation tests, and intramacrophagic leishmanicidal activities linked to the production of NO by monocytes.

The following table shows cellular type responses obtained and the inhibitor role of the serums on parasitic proliferation. Analyses were performed 2 months after the infectious test.

| | | | | | | | % INHIBITION | |
|---|---|---|---|---|---|---|---|---|
| | | CELL-MEDIATED RESPONSES | | | | | OF LEISHMANIAS | |
| | | | | | Leishmanicidal | | PROLIFERATION | |
| | | | Test for | | activity | Dosage | Proliferation | Proliferation |
| | | | lymphoblastic | | of the | of NO | of the | of the |
| | Dogs | IDR | proliferation | | monocytes | (in μM) | promastigotes | amastigotes |
| Dogs | MUMA | + | + 2.1 | (3) | 15.5% | 0.3 | 20% | 15% |
| Controls | LEO | − | + 1.2 | (3.1) | 21.5% | ND | ND | ND |
| Dogs | LOUBARD | + | ++ 2.9 | (3.2) | 58.9% | ND | 50% | 41% |
| immunized | MINA | + | ++ 3.8 | (4.2) | 47.8% | ND | 69% | 52% |
| with ESA | | | | | | | | |
| Dogs | NOUGAT | + | ++ 3.1 | (4.2) | 75.6% | 3.9 | 98% | 54% |
| immunized | MINON | + | +++ 3.5 | (4.5) | 64.1% | 2.8 | 72% | 56% |
| with ESP | | | | | | | | |

Key: IDR: The intra-dermo reaction test is considered positive (+) if the induration is ≧ 5 mm 48 h after intradermal injection; Lymphoblastic proliferation test: The results are expressed by a reading in photon microscope and in stimulation indices (between parentheses, stimulation index of the control + Concanavalin A): +: weak proliferation, ++: medium proliferation, +++: strong proliferation; Leishmanicidal activity of the monocytes: expressed as a percentage of inhibition of the parasitic index; Dosage of NO: Inhibitor role of the serums: results expressed as a percentage inhibition of growth; ND = Not determined

This analysis done on 1 dog for each group (see table above) confirmed that the dogs immunized by ES had very effective antibodies inhibiting both the proliferation of promastigotes and those of the amastigotes compared to controls.

Therefore, it is evident that the pharmaceutical compositions of the invention indeed induce cell-mediated immunity of the Th1 protector type, and induce significant inhibition of the proliferation of Leishmanias by antibodies specific to isotype IgG2.

With reference to FIG. 8, the molecular mass of extracts of promastigotes and/or amastigotes and particular isolates thereof was determined. In FIG. 8, lane marked 1 corresponds to a total TRITON® X-100 (C₁₄H₂₂O(C₂H₄O)₉₋₁₀) polypeptide extract of promastigotes of L. Infantum, lane marked 2 corresponds to isolated excreted-secreted antigen having a molecular mass of about 55.4 kDa, lane marked 3 corresponds to a control, and lane marked 4 corresponds to a total TRITON® X-100 (C₁₄H₂₂O(C₂H₄O)₉₋₁₀) polypeptide extract of amastigotes of L. Infantum.

Determination of media influence on activity of excreted-secreted products

### EXAMPLE 4

Protocol

Clinically healthy Beagles were divided into two groups, each having placebo controls. The 1^{st} group (MPo medium group) received injections of a pharmaceutical composition comprising a product obtained by the method of Example 1 (above) grown on a medium prepared according to Example 6 of 94/26899 WO (the MPo medium). 2^{nd} group (MPm medium group) received injections of a pharmaceutical composition comprising excreted-secreted proteins obtained from promastigotes of Leishmania infantum by the method of Example 1 (above) grown on the MPm medium, as described above.

The injections were administered subcutaneously, four weeks apart. Three injections were administered altogether. The controls received 200 μg of MDO in each injection. The 1^{st} and 2^{nd} groups received 100 μg of excreted-secreted proteins and 200 μg of MDO in the first 2 injections (single dose), and 200 μg of excreted-secreted proteins and 200 μg of MDO in the third injection (double dose).

Results

Observation of local tolerance was carried out daily for 14 days after each injection, and the results are presented below.

| | | First injection | | Second injection | | | Third injection | | |
|---|---|---|---|---|---|---|---|---|---|
| | | (single dose) | | (single dose) | | | (double dose) | | |
| | | MPo | MPm | MPo | | MPm | MPo | | MPm |
| Presence or absence | | medi- | medi- | medi- | | medi- | medi- | | medi- |
| of clinical signs | | um | um | um | | um | um | | um |
| Placebo | + | 1 (0) | 4 (0) | 3 | (1) | 0 | 4 | (1) | 1 (0) |
| dogs | − | 4 | 0 | | | 4 | 1 | | 1 |
| | Total | 5 | 4 | 5 | | 4 | 5 | | 2 |
| Vacci- | + | 9 (2) | 7 (0) | 12 | (7) | 1 (0) | 14 | (7) | 4 (0) |

| | | | | | | | |
|---|---|---|---|---|---|---|---|
| nated | − | 6 | 3 | 3 | 9 | 1 | 4 |
| dogs | Total | 15 | 10 | 15 | 10 | 15 | 8 |

“+”: presence of local reactions (erythema, pain, oedema and/or pruritus) at level 1, 2, 3 or 4; “−”: no visible reaction; numbers w/o parenthesis are count of dogs showing clinical signs of leishmaniasis; numbers in parenthesis are count of dogs showing local reactions at levels 3 or 4.

The results of this study unexpectedly demonstrate a major difference in the toxicity of the pharmaceutical composition comprising excreted-secreted proteins obtained from promastigotes of Leishmania infantum depending on the type of culture medium the parasites were grown in.

An incontestably improved behavior and hence an increased efficacy of the compositions were observed when the excretion-secretion proteins were produced in the modified medium MPm vis-a-vis those produced in the MPo medium. This is explained by a necessary difference in structure of the excretion-secretion proteins depending on the medium in which they were produced.

### EXAMPLE 5

The study is carried out as in Example 4 except MDO is substituted by muramyl dipeptide. Similar results are obtained.

1. A pharmaceutical composition comprising an adjuvant and a mixture of excreted-secreted antigens, said mixture being obtained by a process comprising growing promastigotes of Leishmania sp. in an MPm medium.

2. The pharmaceutical composition of claim 1, wherein said adjuvant is muramyl dipeptide.

3. The pharmaceutical composition of claim 1, wherein said adjuvant is provided in a weight ratio of between 0.5 and 4 with respect to said mixture of excreted-secreted antigens.

4. The pharmaceutical composition of claim 1, provided in a single dose comprising 200 μg of said adjuvant and 100 μg of said mixture of excreted-secreted antigens.

5. The pharmaceutical composition of claim 1, wherein said process for obtaining said mixture of excreted-secreted antigens comprises further the steps of:
(a) filtering a medium in which promastigotes of Leishmania sp. have been grown through a 0.16 μm polyethersulfone microfiltration membrane to obtain a filtrate; and
(b) concentrating said filtrate though a 3 kDa polyethersulfone ultrafiltration membrane.

6. The pharmaceutical composition of claim 1, wherein the mixture of excreted-secreted antigens comprises an excreted-secreted antigen having a molecular mass of about 55.4 kDa.

7. A pharmaceutical composition comprising an adjuvant and a mixture of excreted-secreted antigens, said excreted-secreted antigens being obtained by a process comprising growing amastigotes of Leishmania sp. in an MA1m medium.

8. The pharmaceutical composition of claim 7, wherein said adjuvant is muramyl dipeptide.

9. The pharmaceutical composition of claim 7, wherein said adjuvant is provided in a weight ratio of between 0.5 and 4 with respect to said mixture of excreted-secreted antigens.

10. The pharmaceutical composition of claim 7, provided in a single dose comprising 200 μg of said adjuvant and 100 μg of said mixture of excreted-secreted antigens.

11. The pharmaceutical composition of claim 7, wherein said process for obtaining said mixture of excreted-secreted antigens comprises further the steps of:
(a) filtering a medium in which promastigotes of Leishmania sp. have been grown through a 0.16 μm polyethersulfone microfiltration membrane to obtain a filtrate; and
(b) concentrating said filtrate though a 3 kDa polyethersulfone ultrafiltration membrane.

12. The pharmaceutical composition of claim 7, wherein the mixture of excreted-secreted antigens comprises an excreted-secreted antigen having a molecular mass of about 55.4 kDa.

13. A method for preventing or treating leishmaniasis comprising administering to a patient the pharmaceutical composition of claim 1.

14. A method for preventing or treating leishmaniasis comprising administering to a patient the pharmaceutical composition of claim 7.

15. A method for inducing immunostimulation of lymphocytes of a patient in the need of such immunostimulation comprising administering to the patient the pharmaceutical composition of claim 1.

16. A method for inducing immunostimulation of lymphocytes of a patient in the need of such immunostimulation comprising administering to the patient the pharmaceutical composition of claim 7.

17. A method for inducing immunomodulation of Th1 lymphocytes of a patient in the need of such immunomodulation comprising administering to the patient the pharmaceutical composition of claim 1.

18. A method for inducing immunomodulation of Th1 lymphocytes of a patient in the need of such immunomodulation comprising administering to the patient the pharmaceutical composition of claim 7.

19. The method of claim 13, wherein the leishmaniasis is visceral leishmaniasis.

20. The method of claim 13, wherein the patient is a human or a canine.

21. The pharmaceutical composition of claim 1, wherein the composition contains all excreted-secreted antigens produced in said medium.

22. The method of claim 13, wherein the leishmaniasis is caused by Leishmania infantum.

23. A pharmaceutical composition comprising an adjuvant and a mixture of excreted-secreted antigens, said mixture being obtained by a process comprising growing promastigotes of Leishmania sp. in a culture medium comprising porcine hemin.

24. The pharmaceutical composition of claim 23, wherein the composition contains all excreted-secreted antigens produced in said medium.

25. A method for inducing in a patient preferential expansion of T lymphocytes of the Th1 type with respect to the Th2 type while keeping low levels of IgG2 specific to excreted-secreted antigens comprising administering to the patient the pharmaceutical composition of claim 1.

26. A method for inducing class IgG2 antibodies used as markers of the immune dichotomy Th1/Th2 in a patient in the need thereof, comprising administering to said patient pharmaceutical composition of claim 1.
